(19) United States
(12) Patent Application Publication
(10) Pub. No.: US 2008/0039813 A1
(43) Pub. Date: Feb. 14, 2008

(54) Diaper Accessory

(76) Inventors: Stephenie Ford, Schererville, IN (US); Bryan Ford, Schererville, IN (US)

Correspondence Address: BARNES & THORNBURG LLP, P.O. BOX 2786, CHICAGO, IL, 60690-2786, US

(21) Appl. No.: 11/463,125

(22) Filed: Aug. 8, 2006

(51) Int. Cl. A61F 13/15

(52) U.S. Cl. 604/385.101; 604/385.01; 604/385.11; 604/385.16

(57) ABSTRACT

A diaper accessory including a non-woven fibrous absorptive layer adapted to absorb and retain waste expelled by an infant that is not captured by the disposable diaper. The diaper accessory also includes a water resistant layer positioned adjacent to the non-wove fibrous layer. Attached to the water resistant layer is an adhesive layer that is used to secure the diaper pad to the article of clothing worn by the infant. The adhesive layer is covered by a release liner. The release line is removed prior to using the diaper pad to expose the adhesive layer. The diaper pad can be coupled to the disposable diaper or can be used in combination with a disposable diaper to capture waste created by the infant. The diaper pad is modular, in that it includes a plurality of removable strips that allow portions of the diaper pad to be removed to allow for sizing of the diaper pad to the infant.

### BACKGROUND

The present disclosure relates to diapers, and, in particular, to disposable diapers that are used with infants. More particularly, the present disclosure relates to accessories for use with disposable diapers to prevent infants from soiling their clothing. Diapers typically are disposable, are worn by infants to absorb and retain waste produced by the infant. Disposable diapers, under certain conditions, cannot contain the waste produced by the infant resulting in the garments worn by the infant becoming saturated with waste. Undesired leakage of the diaper requires replacement of the child's outfit. This situation occurs most often in infants that are breastfed.

### SUMMARY

A diaper accessory in accordance with the present disclosure in the form of a diaper pad includes a non-wove fibrous absorptive layer adapted to absorb and retain waste expelled by an infant that is not captured by the disposable diaper. The diaper accessory also includes a water resistant layer positioned adjacent to the non-woven fibrous layer. Attached to the water resistant layer is an adhesive layer that is used to secure the diaper pad to the article of clothing worn by the infant. The adhesive layer is covered by a release liner. The release liner is removed prior to using the diaper pad to expose the adhesive layer. The diaper pad can be coupled to the disposable diaper or can be used in combination with a disposable diaper to capture waste created by the infant and not contained by the disposable diaper. The diaper pad is modular, in that it includes a plurality of removable strips that allow portions of the diaper pad to be removed to allow for sizing of the diaper pad to the infant.

Additional features of the present disclosure will become apparent to those skilled in the art upon consideration of the following detailed description of illustrative embodiments exemplifying the best mode for carrying out the disclosure as presently perceived.

### BRIEF DESCRIPTION OF THE DRAWINGS

The detailed descrition particularly refers to the accompanying figures in which:

FIG. 1 is a perspective view of a disposable diaper showing a diaper pad, in accordance with the present disclosure, extending upwardly from the rear portion of the diaper to capture waste that is not contained by the disposable diaper.

FIG. 2 is atop view of the disposable diaper of FIG. 1 showing the interior surface of the disposable diaper and the diaper pad coupled to the rear portion of the disposable diaper; and

FIG. 3 is a perspective view of the diaper pad showing a portion of the release liner being peeled away from the diaper pad, and also showing the perforated lines to allow removal of portions of the diaper pad to permit proper sizing for the infant.

### DETAILED DESCRIPTION

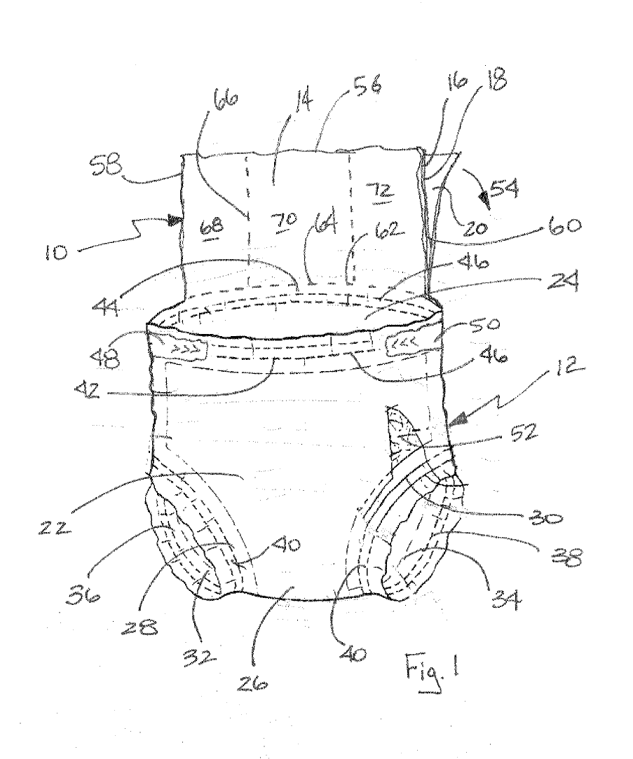

A diaper pad 10 is adapted to be used in combination with a diaper 12. The diaper 12 includes a front portion 22, a rear portion 24, and a bridge portion 26, as sown, for example, in FIG. 1. The front portion 22 of diaper 12 includes first and second arcuate walls 28, 30, that form the front portion of leg openings 32, 34. Leg openings 32, 34 permit the passage of the infant's legs. Leg openings 32 and 34 are defined by arcuate wall sections 36, 38. Leg openings 32, 34, formed by front and rear portions 22, 24 also include elastic 40 to secure diaper 12 around the legs of the infant to prevent leakage around openings 32, 34. While the diaper pad is used with a disposable diaper, it should be understood that the pad 10 can be used with other forms of diapers as long as they are used in connection with the pad 10.

Diaper 12 also includes a front waistband portion 42 and a rear waistband portion 44. Waistband portions 42, 44 include elastic material 46 to seal front and rear portions 22, 24 against the abdomen of the child as shown, for example, in FIG. 1. Rear portion 24 of diaper 12 includes first and second retention tabs 48, 50 that are used to secure rear portion 24 of the diaper 12 to front portion 22. Retention tabs 48, 50 can be secured by use of an adhesive or by use of a hook and loop type fastener. Diaper 12 preferably includes a non-woven fibrous and moisture absorbing inner layer 52 to absorb and retain waste produced by the infant.

The rear waistband portion 44 of rear portion 24 includes diaper pad 10 as shown, for example, in FIG. 1. Diaper pad 10 acts as a leak retention pad to absorb bodily fluids that escape from the diaper 12. Diaper pad 10 includes a non-woven fibrous layer 14 that is secured to a water resistant layer 16. The non-woven fibrous layer 14 may include an embossed pattern to aid in trapping the fluid. The diaper pad 10 is from about three inches to about six inches wide. While a non-woven fibrous material is used, it is contemplated that other absorbent materials can be used including woven material such as cotton or other materials. Water resistant layer 16 includes an adhesive layer 18 that is covered by a release liner 20. To use diaper pad 10 release layer 20 is peeled away from adhesive liner 18 in direction 54. This allows the adhesive layer 18 to be adhered to the clothing worn by the infant.

Diaper pad 10 includes a first end wall 56 and a pair of opposing side walls 58, 60. Diaper pad 10 also includes end wall 62 that includes a plurality of perforations 64 to permits diaper pad 10 to be separated from diaper 12 along perforation 64. Diaper pad 10 is preferably made from a biodegradable material. Diaper pad 10 also includes vertically oriented perforations 66 to allow portions 68, 70, 72 to be selectively removed from diaper pad 10 to allow proper sizing of the pad for the infant. The perforations form weakened zones to aid in removal of portions 68, 70, 72. While three portions are shown, it is contemplated that the diaper pad 10 can be designed with multiple portions to permit custom tailoring of diaper pad 10 to the dimensions of the infant.

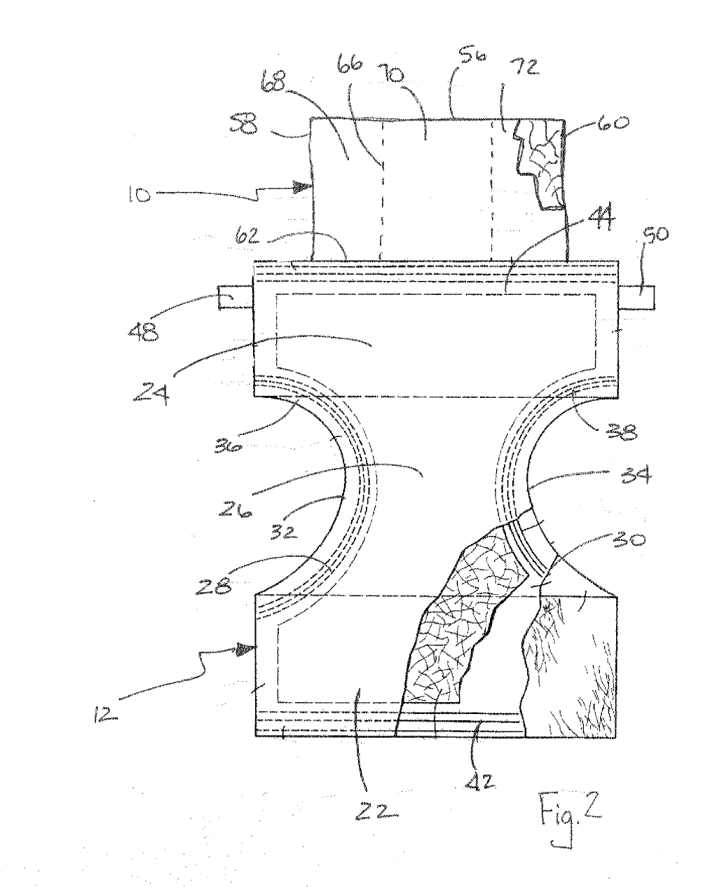

Front and rear portions 22, 24 of diaper 12 are interconnected by bridge portion 26. Diaper pad 10 is connected to rear waistband portion 44 of diaper 10. Diaper pad 10 is adapted to extend outwardly from rear waistband portion 44 so that when the infant is positioned on the rear portion 24 of diaper 12, diaper pad 10 is properly positioned adjacent the lower back portion of the infant. Also shown in FIG. 2 are retention tabs 48, 50, and arcuate wall sections 28, 30, 36, 38 of leg openings 32, 34. When the lower portion of the infant's torso is positioned on rear portion 24 of diaper 12, the front portion 22 is folded upward around the front of the infant and retention tabs 48, 50 are positioned on front portion 22 of diaper 12. In the event that rear waistband portion 44 cannot contain waste created by the infant, diaper pad 10 acts as a barrier between the infant and the article of clothing worn by the infant to prevent soiling of the clothing.

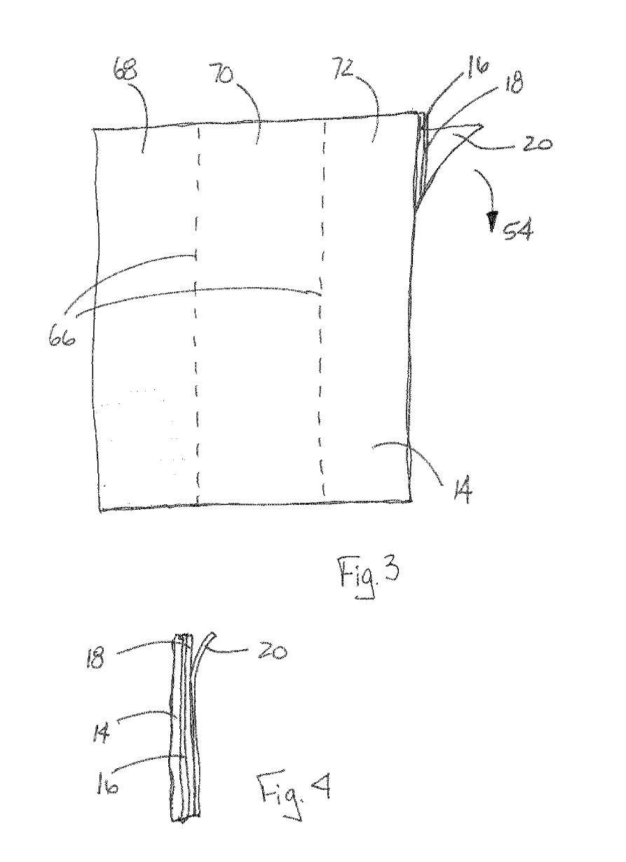

Diaper pad 10 can also be used independently of, but in combination with, diaper 12. Diaper pad 10, as shown, for example, in FIG. 3 includes a non-woven fibrous layer 14 coupled to a water resistant layer 16. The non-woven fibrous layer 14 is made from a water absorbing material such as cellulose fiber, cotton or other moisture absorbent material. Water resistant layer 16 is secured to non-woven fibrous layer 14 by use of an adhesive or is bonded sheet by use of a polymer tie layer.

Water resistant layer 16, as shown, for example, in FIGS. 3 and 4, is preferably made out of a polymer such as polypropylene polyethylene or from other polymers designed to prevent the passage of moisture. Water resistant layer 16 includes an adhesive layer 18 that allows diaper pad 10 to be secured to the inside surface of the back portion of an infant garment to secure the position of the diaper pad 10 with respect to the garment. The use of adhesive layer 18 prevents diaper pad 10 from bunching up under the infant's clothing. The diaper pad 10, when used as a separate article from diaper 12, is positioned adjacent the rear waistband portion 44 of diaper 12 to provide a leak-proof barrier between the child's back and the article of clothing that the infant is wearing. Diaper pad 10 also includes vertically oriented perforations 66 to allow diaper pad 10 to be sized to fit the width of the infant's back. This allows a single diaper pad to be produced that can be used for infants of various sizes.

In use, diaper 12 is placed on and fastened around the infant so that the front portion 22 is positioned adjacent the front side of the child and rear portion 24 is positioned adjacent the rear side of the child. Once the front and rear portions 22, 24 are in position, retention tabs 48, 50 from rear portion 24 are secured to front portion 22. With diaper 12 in position, diaper pad 10 is adjusted so that it is positioned between the back of the infant and the article of clothing worn by the infant. To secure diaper pad 10 to the infant's garment, release liner 20 is removed from water resistant layer 16 to expose adhesive layer 18. This arrangement prevents unwanted movement of diaper pad 10 with respect to the infant's garment. If the diaper 12 becomes soiled and the parent wishes to reuse the diaper pad portion 10 of disposable diaper 12, diaper pad 10 can be separated from disposable diaper 12 along perforation 64. If diaper pad 10 is used as an accessory to a standard disposable diaper, then the diaper pad is positioned adjacent the rear waistband portion 44 of the diaper between the back of the infant and the infant's garment. It is preferable that a portion of diaper pad 10 overlap rear waistband portion 44 to ensure that waste does not escape between diaper pad 10 and diaper 12. To maintain the orientation of the diaper pad when used alone, release liner 12 is removed from adhesive layer 18 and diaper pad 10 is applied to the inside surface of the infant's garment adjacent lower portion of the child's back. If diaper pad 10 is too wide for a given infant, portions of diaper pad 10 can be removed along perforations 66 to properly fit diaper pad 10 to the infant. Upon changing an infant's diaper, the soiled diaper 12 and diaper pad are removed from the infant, placed in a waste receptacle and a new diaper and diaper pad are applied.

Various features of the invention have been particularly shown and described in connection with the illustrated embodiment of the invention, however, it must be understood that these particular arrangements merely illustrate, and that the invention is to be given its fullest interpretation.

What is claimed is:

1. A disposable diaper having a lead retention pad that is capable of preventing an infant's waste from leading onto garments worn by the infant, the disposable diaper comprising:
a diaper body having a front panel adapted to be positioned adjacent a front lower portion of the infant's torso;
a rear panel connected to the front panel by a bridge panel, the rear panel adapted to be positioned adjacent a rear lower portion of the infant's torso, the diaper including retention tabs that are adapted to couple the front panel to the rear panel to secure the disposable diaper to the infant, the diaper also including at least one absorbent layer for retaining the infant's waste;
the leak retention pad secured to an upper edge of the rear panel, the leak retention pad having at least one absorbent layer and a moisture resistant layer, the leak retention pad being adapted to extend outwardly from the upper edge of the rear panel toward the infant's head when positioned between the infant's back and the garment worn by the infant;
wherein the leak retention pad retains waste that escape from the upper edge of the rear panel to prevent the waste from leaking into the garment.

2. The disposable diaper of claim 1, wherein the leak retention pad includes an embossed pattern to aid in trapping the waste.

3. The disposable diaper of claim 1, wherein the leak retention pad is detachable from the disposable diaper.

4. The disposable diaper of claim 1, wherein the absorbent layer of the leak retention pad is made from a non-woven fibrous absorbent material.

5. The disposable diaper of claim 1, wherein portions of the leak retention pad can be removed to size the leak retention pad to the infant.

6. The disposable diaper of claim 1, wherein the retention pad is from about 4 inches wide to about six inches wide.

7. The disposable diaper of claim 1, wherein the retention pad is from about 4 inches long to about six inches long.

8. A disposable modular leak retention pad that is capable of preventing an infant's waste from leaking onto garments worn by the infant, the disposable modular leak retention pad comprising:
at least one layer of non-wove fibrous absorbent material, said at least one layer of non-woven fibrous absorbent material adapted to partially absorb and retain the waste of the infant and not cause irritation to the infant's skin;
a moisture impervious layer attached to the at least one layer of non-woven fibrous absorbent material, the moisture impervious layer adapted to prevent the passage of moisture through the moisture impervious layer;
an adhesive layer attached to one side of the moisture impervious layer;
a release liner releasably secured to the adhesive layer, the release liner adapted to be selectively removable from the adhesive layer to expose the adhesive layer, to allow the adhesive layer to be adhered to the garment of the infant;
wherein the at least one layer of non-woven fibrous absorbent material and moisture impervious layer included weakened zones forming tear lines to allow portions of the pad to be removed so that the width of the pad can be decreased to permit the pad to be sized to fit the width of the infant's back.

9. The disposable modular leak retention pad of claim 8, wherein the at least one layer of non-woven fibrous absorbent material includes an embossed pattern to aid in trapping the waste.

10. The disposable modular leak retention pad of claim 8, wherein the pad is made from biodegradable material.

11. The disposable modular leak retention pad of claim 8, wherein the release liner also includes a weakened zone to form tear lines.

12. The disposable modular leak retention pad of claim 8, wherein the pad can be removed from and reapplied to the garment.

13. A method of using a retention pad to retain infant waste, that has escaped from a rear portion of a waistband of a disposable diaper, to prevent soiling of a back portion of a garment worn by an infant, the retention pad having at least one layer of non-woven fibrous absorbent material, a moisture impervious layer attached to the at least one layer of non-woven fibrous absorbent material, and adhesive layer attached to one side of the moisture impervious layer, and a release liner releasably secured to the adhesive layer, the method comprising the steps of:
removing the release liner from the adhesive layer of the retention pad so that the adhesive layer of the retention pad is exposed;
applying the adhesive layer of the retention pad to the back portion of the garment worn by the infant, adjacent the waistband of the disposable diaper;
positioning the retention pad against the back of the infant such that a portion of the retention pad overlaps the waistband of the disposable diaper.

14. The method of using a retention pad to retain infant waste of claim 13, further comprising the steps of removing the retention pad from the garment in the event it becomes soiled.

15. The method of using a retention pad to retain infant waste of claim 13, wherein the at least one layer of non-woven fibrous absorbent material including an embossed pattern to aid in trapping waste.

16. The method of using a retention pad to retain infant waste of claim 13, wherein the retention pad is from about 4 inches wide to about six inches wide.

17. The method of using a retention pad to retain infant waste of claim 13, wherein the retention pad is from about 4 inches long to about six inches long.
